(19) United States
(12) Patent Application Publication
(10) Pub. No.: US 2023/0056360 A1
(43) Pub. Date: Feb. 23, 2023

(54) BLOOD-BRAIN-BARRIER SYSTEMS

(71) Applicant: B. G. NEGEV TECHNOLOGIES AND APPLICATIONS LTD., AT BEN-GURION UNIVERSITY, Beer Sheva (IL)

(72) Inventors: Gad David VATINE, Ramat Gan (IL); Roman KHOURIN, Rishon LeTsiyon (IL)

(21) Appl. No.: 17/796,018

(22) PCT Filed: Jan. 28, 2021
(86) PCT No.: PCT/IL2021/050101

§ 371 Date: Jul. 28, 2022

Related U.S. Application Data
(60) Provisional application No. 62/967,213, filed on Jan. 29, 2020.

(51) Int. Cl. G01N 33/50; C12M 1/12; C12M 1/34; C12N 5/071

(52) U.S. Cl. CPC G01N 33/5032; C12M 25/04; C12M 41/46; G01N 33/5082; C12N 5/0697; C12N 2506/45; C12N 2500/90; C12N 2502/081

(57) ABSTRACT

Disclosed herein is a device comprising a microelectrode comprising cells cultured on a surface of the microelectrode and a porous membrane comprising an upper surface comprising cultured cells. Further, devices and methods for in in-vitro models of the blood-brain barrier (BBB) and for modeling the transport across this barrier are disclosed.

### CROSS-REFERENCE TO RELATED APPLICATIONS

This application claims the benefit of priority to U.S. Provisional Application No. 62/967,213 titled “BLOOD-BRAIN-BARRIER SYSTEMS” filed Jan. 29, 2020, the contents of which are incorporate herein by reference in their entirety.

### FIELD OF INVENTION

The present invention, in some embodiments, is directed to blood-brain barrier modeling, and includes brain region-specific neurovascular unit (NVU) platforms.

### BACKGROUND

The blood brain barrier (BBB) is a highly selective barrier that shields the central nervous system (CNS) from a range of factors in the circulating blood that could disrupt delicate brain functioning. The BBB is formed as a multicellular neurovascular unit (NVU) in which pericytes, astrocyte end-feet and neuronal axons come in direct contact with brain microvascular endothelial cells (BMECs). In turn, BMECs form specialized barrier properties created by tight junctions (TJs), which limit the paracellular passage of molecules, and by polarized efflux transporters, which form a transport barrier by pumping molecules back into the blood. As a result, the BBB facilitates the passage of specific nutrients and metabolic necessities into the brain but restricts the entry of neurotoxic agents and most drugs. Thus, the BBB constitutes a major obstacle to the delivery of biopharmaceuticals into the brain, and less than 5% of FDA-approved small molecules can even reach the brain. A major challenge in the field is the lack of robust BBB models that faithfully predict the penetrability of candidate drugs to the human brain.

Animal models have been widely used to study BBB penetrance and molecular mechanisms that are involved in BBB development. These models are readily available, faithfully represent the complex in vivo environment, possess physiological barrier properties, and importantly, provide the opportunity for applying transgenic models. However, discrepancies between species and the poor ability of current models to predict drug delivery into the human CNS severely question their relevance. Thus, human-based in vitro models are critically needed to study the human BBB and for drug development.

To faithfully mimic the in vivo BBB, in vitro models must display physiologically relevant BBB properties, including low permeability, physiologically relevant trans-endothelial electrical resistance (TEER), and polarized efflux-pump activity, and they must also include representative expression of functional transport systems. Typically, human in vitro models are comprised of endothelial cells that are co-cultured on a semi-permeable membrane with combinations of other cells of the NVU to enhance BBB properties. Recent developments in protocols to differentiate induced pluripotent stem cells (iPSCs) into brain microvascular endothelial cells (BMECs) were introduced as an attractive source for in vitro human BBB models. iPSC-derived BMECs (iBMECs) are highly scalable and demonstrate crucial characteristics of the human BBB, including physiological barrier properties that are as high as values measured in vivo (5,000 Ωcm²), polarization, and expression of functional efflux pumps. These features make iBMEC-based models a robust source for modeling the human BBB.

However, a major limitation of in vitro models of the BBB is that they fail to represent the heterogeneity of the BBB in different areas of the human central nervous system (CNS). Here, the inventors report the generation of in vitro iBMEC-based NVU platforms designed to test the CNS-penetrability in a brain-region specific manner.

Similarly to the variation in capillary functions across different organs, the functional properties vary in different regions of the CNS. Circumventricular organs (CVOs) are structures in the brain that are characterized by their extensive and highly permeable capillaries in contrast to capillaries in the rest of the CNS where there exists a functional BBB. While these variations in the function of brain capillaries across different CNS regions are well established, the heterogeneous nature of brain capillaries within different areas of the brains in which the BBB is functional has been largely overlooked. BBB heterogeneity has been observed in various aspects: morphological differences, differences in astrocytes, regional differences in pericytes, differences in cerebral endothelial cells, and permeability properties. Altogether, these variations strongly imply that the differential interactions in brain region specific NVUs may result in variations in the region-specific BBB performance. Having the ability to model these variations will facilitate understanding the underlying mechanisms and importantly, to predict CNS penetrability into specific regions of the brains, which are often specifically affected in neurological diseases. There is still a great need to direct therapies specifically to these regions.

### SUMMARY

The present invention, in some embodiments, is based in part, on the findings that iBMECs can be successfully used for the development of brain region-specific NVU platforms.

In one aspect of the invention, there is provided a device comprising: i. a microelectrode comprising cells cultured on a surface of the microelectrode; and ii. a porous membrane comprising an upper surface comprising cultured cells and a lower surface; wherein: the porous membrane is positioned above the microelectrode and the cells cultured on the microelectrode are facing the lower surface of the porous membrane.

In some embodiments, the lower surface is devoid of the cultured cells of the upper surface.

In some embodiments, the cells cultured on the microelectrode comprise at least 90% neural cells.

In some embodiments, the cells cultured on the microelectrode comprise at least 90% neural cells from a single brain region.

In some embodiments, the neural cells are selected from the group consisting of: cortex neural cells, striatum neural cells, hippocampus neural cells, cerebellum neural cells, and spinal cord neural.

In some embodiments, the cultured cells on the porous membrane comprise at least 90% endothelial cells.

In some embodiments, the cultured cells on the porous membrane comprise at least 90% brain microvascular endothelial cells.

In some embodiments, the cultured cells on the porous membrane comprise at least 90% induced pluripotent stem cell-derived brain microvascular endothelial cells (iBMECs).

In some embodiments, the cells cultured on the microelectrode comprise glial cells.

In some embodiments, the microelectrode comprises a microelectrode array (MEA) plate.

In some embodiments, the porous membrane is characterized by an electrical resistance of at least 5000 Ω×cm².

In some embodiments, the porous membrane and the microelectrode are inside a chamber comprising a fluid.

In some embodiments, the microelectrode are each inside a separate chamber and in fluid communication.

In some embodiments, the fluid comprises a cell culture medium.

In another aspect of the invention, there is provided a method for measuring resistance of a blood-brain-barrier (BBB), comprising measuring resistance of the porous membrane within the device of the present invention, thereby measuring the resistance of the BBB.

In another aspect of the invention, there is provided a method for measuring resistance of a blood-brain-barrier (BBB) in a single brain region, comprising measuring resistance of the membrane within the device of the present invention, thereby measuring the resistance of the BBB in a single brain region.

In another aspect of the invention, there is provided a method for determining an ability of a compound to affect permeability of a BBB, comprising administering the compound in an upper portion of the device of the present invention, and then measuring resistance of the porous membrane within the device of the present invention, thereby determining the ability of a compound to affect the permeability of the BBB.

In another aspect of the invention, there is provided a method for determining an ability of a compound to affect permeability of a BBB in a single brain region, comprising administering the compound in an upper portion of the device of the present invention, and then measuring resistance of the membrane within the device of the present invention, thereby determining the ability of a compound to affect the permeability of the BBB in a single brain region.

In another aspect of the invention, there is provided a method for determining an ability of a compound to traverse a BBB, comprising administering the compound in an upper portion of the device of the present invention, and then measuring amount of the compound in the lower portion of the device of the present invention, thereby determining the ability of a compound to traverse the BBB.

In another aspect of the invention, there is provided a method for determining an ability of a compound to traverse a BBB in a single brain region, comprising administering the compound in an upper portion of the device of the present invention, and then measuring amount of the compound in the lower portion of the device of the present invention, thereby determining the ability of a compound to traverse the BBB in a single brain region.

In another aspect of the invention, there is provided a method for determining an ability of a compound to modulate neuronal activity across a BBB, comprising administering the compound in an upper portion of the device of the present invention, and then measuring an action potential of the neural cells of the present invention, thereby determining the ability of a compound to modulate neuronal activity across the BBB.

In another aspect of the invention, there is provided a method for determining an ability of a compound to modulate neuronal activity across a BBB in a single brain region, comprising administering the compound in an upper portion of the device of the present invention, and then measuring an action potential of the neural cells of the present invention, thereby determining the ability of a compound to modulate neuronal activity across the BBB in a single brain region.

In another aspect of the invention, there is provided a method for determining a neurotoxic potential of a compound across a BBB, comprising administering the compound in an upper portion of the device of the present invention, and then assessing cell death of the neural cells of the present invention, thereby determining the neurotoxic potential of a compound across the BBB.

In another aspect of the invention, there is provided a method for determining a neurotoxic potential of a compound across a BBB in a single brain region, comprising administering the compound in an upper portion of the device of the present invention, and then assessing cell death of the neural cells of the present invention, thereby determining the neurotoxic potential of a compound across the BBB in a single brain region.

In another aspect of the invention, there is provided a method for generating modified iBMECs, comprising culturing iBMECs for at least 10 hours in the presence of a composition comprising an extra-cellular environment of neural cells or cultured within an upper portion of the device of the present invention.

In another aspect of the invention, there is provided a method for differentiating induced pluripotent stem cells (iPSCs) to brain microvascular endothelial cells (BMECs), wherein the iPSCs are cultured in serum free medium, thereby differentiating the iPSCs to BMEC s.

In some embodiments, the BMECs are cultured within an upper portion of the device of the present invention, and the electrical resistance of the membrane is at least 5000 Ω×cm².

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-B present a schematic illustration depicting a non-limiting exemplary configuration of the disclosed device;

FIGS. 2A-C present images of primary neural cultures from different regions of rat brain: primary neural cultures were derived from striatum, hippocampus, cortex and cerebellum of E18 rat embryos. Primary cultures expressed the neuronal marker βIII-tubulin and the glial marker Glial fibrillary acidic protein (GFAP) (FIG. 2A); primary neural cells grown on a microelectrode array (MEA, Axion BioSystems) plate (FIG. 2B); a graph of representative action potential recorded from a cortical primary neuron (FIG. 2C);

FIG. 3 presents a differentiation scheme of human induced pluripotent stem (iPSCs) into BMECs; at day 9 post differentiation, iBMECs inserts were placed on top of two weeks old rat primary neural cultures derived from the cortex, striatum, hippocampus cerebellum and spinal cord;

FIG. 4 presents a graph of transendothelial electrical resistance (TEER) measurements of iBMECs that were co-cultured over primary rat brain region-specific neural cell cultures: iBMECs on a Transwell insert cultured alone (black bars) or co-cultured on top of primary neural cultures derived from various region of E18 rat brain including cortex, hippocampus, striatum, cerebellum and spinal cord. All co-cultured displayed a significant increase in TEER compared to iBMECs cultured alone (ANOVA P<0.001). Significant differences were also observed between different co-cultures (* p<0.05, **p<0.01);

FIGS. 5A-F present bar graphs of the permeability of molecules across iBMECs in monocultures or in NVUs of the different brain regions: permeability of dextran-TRITC 70 kDa (FIG. 5A), dextran-FITC 20 kDa (FIG. 5B), dextran-FITC 4 kDa (FIG. 5C), glucose analogue fluorescent deoxyglucose derivative (2-NBDG) (FIG. 5D), sodium fluorescein 332 Da (FIG. 5E) and the efflux pump substrate Rhodamine (FIG. 5F). Results were derived from two independent experiments performed in triplicates. One-way ANOVA followed by Tukey test to adjust for multiple comparisons n=5 *P<0.05 **P<0.01. Error bars represent SEM.

### DETAILED DESCRIPTION

The present invention is directed to a device for use in-vitro models of the blood-brain barrier (BBB) and modeling the transport across BBB barrier. In some embodiments, the device disclosed herein allows for the test and study of the permeability and transport across BBB barrier in a brain-region specific manner.

The present invention is also directed to a method for mimicking or modeling the BBB in-vitro. In some embodiments, the method is for in-vitro mimicking or modeling the BBB of a specific brain region.

The present invention is based, in part, on the finding that iBMECs can be successfully used for the development of brain region-specific NVU platforms.

In another aspect, the present invention is based in part, on the finding that using the device of the present invention and applying the methods of the present invention it is possible to detect different permeability between the various neurovascular units. In some embodiments, neural cells increase the barrier functions in iBMECs.

In some embodiments, the present invention provides a device comprising a microelectrode, a porous membrane on top of the microelectrode and cells cultured on the microelectrode and the membrane. In some embodiments, the present invention provides a device comprising a microelectrode, a porous membrane on top of the microelectrode and cells cultured separately on the microelectrode and the membrane. In some embodiments, the present invention provides that the cells cultured on top of the microelectrode and the cells cultured on the membrane are different. In some embodiments, the present invention provides that the cells cultured on top of the microelectrode are devoid of endothelial cells. In some embodiments, the present invention provides that the cells cultured on top of the membrane are devoid of neuronal or nervous system cells. In some embodiments, the devise is a Transwell-like system.

### The Device

According to an aspect of the present invention, there is provided a device comprising: a microelectrode comprising cells cultured on a surface of the microelectrode; and a porous membrane comprising an upper surface comprising cultured cells and a lower surface; wherein the porous membrane is positioned above the microelectrode and the cells cultured on a surface of the microelectrode are facing the lower surface of the porous membrane. In some embodiments, the upper surface is an outer surface.

In some embodiments, the lower surface is devoid of the cultured cells of the upper surface.

In some embodiments, there is provided a device comprising a microelectrode comprising cells cultured on a surface of the microelectrode; and a porous membrane comprising cells cultured on a surface of the porous membrane. In some embodiments, the porous membrane is positioned in a parallel plane above the microelectrode. In some embodiments, the cells cultured on a surface of the porous membrane are facing the opposite side of the microelectrode.

In some embodiments, the device is a stackable transwell-like device. In some embodiments, the device comprises a top transwell insert and a bottom transwell insert. In some embodiments, the top transwell insert, stacks above the bottom transwell insert. In some embodiments, the top transwell insert comprises a porous membrane as described herein. In some embodiments, the bottom transwell comprises a microelectrode as described herein.

In some embodiments, the microelectrode and the porous membrane are in contact. In some embodiments, the microelectrode and lower surface of the porous membrane are in contact. In some embodiments, the microelectrode and the porous membrane are in two parallel planes at a distance from each other. In some embodiments, the distance between the microelectrode and the porous membrane can be pre-determined and varied. In some embodiments, the cells cultured on the microelectrode are not in physical contact with the cells cultured on the porous membrane.

In some embodiments, the cells cultured on the microelectrode are in a different portion of the device that the cells cultured on the porous membrane. In some embodiments, the cells cultured on the microelectrode are in fluid communication with the cells cultured on the porous membrane.

In some embodiments, the cells cultured on the microelectrode comprise at least 90%, at least 91%, at least 92%, at least 93%, at least 94%, at least 95%, at least 96%, at least 97%, at least 98%, or at least 99% neural cells, including any value therebetween. Each possibility represents a separate embodiment of the invention.

In some embodiments, the cells cultured on the microelectrode comprise between 90% and 100%, between 91% and 100%, between 95% and 100%, between 97% and 100%, between 90% and 99%, between 91% and 99%, between 95% and 99%, between 97% and 99%, between 90% and 98%, between 91% and 98%, between 95% and 98%, between 97% and 98%, between 90% and 95%, or between 91% and 95%, neural cells, including any range therebetween. Each possibility represents a separate embodiment of the invention.

In some embodiments, the cells cultured on the microelectrode comprise at least 90%, at least 91%, at least 92%, at least 93%, at least 94%, at least 95%, at least 96%, at least 97%, at least 98%, or at least 99% neural cells from a single brain region, including any value therebetween. Each possibility represents a separate embodiment of the invention.

In some embodiments, the cells cultured on the microelectrode comprise between 90% and 100%, between 91% and 100%, between 95% and 100%, between 97% and 100%, between 90% and 99%, between 91% and 99%, between 95% and 99%, between 97% and 99%, between 90% and 98%, between 91% and 98%, between 95% and 98%, between 97% and 98%, between 90% and 95%, or between 91% and 95% neural cells from a single brain region, including any range therebetween. Each possibility represents a separate embodiment of the invention.

In some embodiments, the neural cells are selected from the group consisting of: cortex neural cells, striatum neural cells, hippocampus neural cells, cerebellum neural cells, and spinal cord neural.

In some embodiments, the cells cultured on the porous membrane comprise at least 90%, at least 91%, at least 92%, at least 93%, at least 94%, at least 95%, at least 96%, at least 97%, at least 98%, or at least 99% endothelial cells, including any value therebetween. Each possibility represents a separate embodiment of the invention.

In some embodiments, the cells cultured on the porous membrane comprise between 90% and 100%, between 91% and 100%, between 95% and 100%, between 97% and 100%, between 90% and 99%, between 91% and 99%, between 95% and 99%, between 97% and 99%, between 90% and 98%, between 91% and 98%, between 95% and 98%, between 97% and 98%, between 90% and 95%, or between 91% and 95% endothelial cells, including any range therebetween. Each possibility represents a separate embodiment of the invention.

In some embodiments, the cells cultured on the upper surface of the porous membrane comprise at least 90%, at least 91%, at least 92%, at least 93%, at least 94%, at least 95%, at least 96%, at least 97%, at least 98%, or at least 99% endothelial cells, including any value therebetween. Each possibility represents a separate embodiment of the invention.

In some embodiments, the cells cultured on the upper surface of the porous membrane comprise between 90% and 100%, between 91% and 100%, between 95% and 100%, between 97% and 100%, between 90% and 99%, between 91% and 99%, between 95% and 99%, between 97% and 99%, between 90% and 98%, between 91% and 98%, between 95% and 98%, between 97% and 98%, between 90% and 95%, or between 91% and 95% endothelial cells, including any range therebetween. Each possibility represents a separate embodiment of the invention.

In some embodiments, the cells cultured on the porous membrane comprise at least 90%, at least 91%, at least 92%, at least 93%, at least 94%, at least 95%, at least 96%, at least 97%, at least 98%, or at least 99% brain microvascular endothelial cells, including any value therebetween. Each possibility represents a separate embodiment of the invention.

In some embodiments, the cells cultured on the porous membrane comprise between 90% and 100%, between 91% and 100%, between 95% and 100%, between 97% and 100%, between 90% and 99%, between 91% and 99%, between 95% and 99%, between 97% and 99%, between 90% and 98%, between 91% and 98%, between 95% and 98%, between 97% and 98%, between 90% and 95%, or between 91% and 95% brain microvascular endothelial cells, including any range therebetween. Each possibility represents a separate embodiment of the invention.

In some embodiments, the cells cultured on the porous membrane comprise at least 90%, at least 91%, at least 92%, at least 93%, at least 94%, at least 95%, at least 96%, at least 97%, at least 98%, or at least 99% induced pluripotent stem cell-derived brain microvascular endothelial cells (iBMECs), including any value therebetween. Each possibility represents a separate embodiment of the invention.

In some embodiments, the cells cultured on the porous membrane comprise between 90% and 100%, between 91% and 100%, between 95% and 100%, between 97% and 100%, between 90% and 99%, between 91% and 99%, between 95% and 99%, between 97% and 99%, between 90% and 98%, between 91% and 98%, between 95% and 98%, between 97% and 98%, between 90% and 95%, or between 91% and 95% induced pluripotent stem cell-derived brain microvascular endothelial cells (iBMECs), including any range therebetween. Each possibility represents a separate embodiment of the invention.

In some embodiments, the cells cultured on the upper surface of the porous membrane comprise at least 90%, at least 91%, at least 92%, at least 93%, at least 94%, at least 95%, at least 96%, at least 97%, at least 98%, or at least 99% brain microvascular endothelial cells, including any value therebetween. Each possibility represents a separate embodiment of the invention.

In some embodiments, the cells cultured on the upper surface of the porous membrane comprise between 90% and 100%, between 91% and 100%, between 95% and 100%, between 97% and 100%, between 90% and 99%, between 91% and 99%, between 95% and 99%, between 97% and 99%, between 90% and 98%, between 91% and 98%, between 95% and 98%, between 97% and 98%, between 90% and 95%, or between 91% and 95% brain microvascular endothelial cells, including any range therebetween. Each possibility represents a separate embodiment of the invention.

In some embodiments, the cells cultured on the upper surface of the porous membrane comprise at least 90%, at least 91%, at least 92%, at least 93%, at least 94%, at least 95%, at least 96%, at least 97%, at least 98%, or at least 99% induced pluripotent stem cell-derived brain microvascular endothelial cells (iBMECs), including any value therebetween. Each possibility represents a separate embodiment of the invention.

In some embodiments, the cells cultured on the upper surface of the porous membrane comprise between 90% and 100%, between 91% and 100%, between 95% and 100%, between 97% and 100%, between 90% and 99%, between 91% and 99%, between 95% and 99%, between 97% and 99%, between 90% and 98%, between 91% and 98%, between 95% and 98%, between 97% and 98%, between 90% and 95%, or between 91% and 95% induced pluripotent stem cell-derived brain microvascular endothelial cells (iBMECs), including any range therebetween. Each possibility represents a separate embodiment of the invention.

In some embodiments, the cells cultured on the microelectrode comprise glial cells.

In one embodiment, the cells include any combination of cells as provided herein. In one embodiment, the cells include any combination of cell types as provided herein. In one embodiment, the cells include any combination of cell types derived from a single brain region as provided herein. In one embodiment, the cells are derived from a single brain region as provided herein. In one embodiment, a combination of cells is derived from a single brain region as provided herein.

In some embodiments, the porous membrane and the microelectrode are inside a chamber. In some embodiments, the porous membrane and the microelectrode are each inside a separate chamber and in fluid communication.

In some embodiments, each chamber comprises an opening configured to receive a fluid. In some embodiments, the chamber comprises a fluid. In some embodiments, the fluid comprises a cell culture medium.

As used herein, “cell culture medium”, refers to a solution containing sufficient nutrients to promote the growth of cells in a culture. Typically, these solutions contain essential amino acids, non-essential amino acids, vitamins, energy sources, lipids and/or trace elements. The medium can also contain other adjuvants such as hormones, growth factors and growth inhibitors. The requirements for these components vary among cell lines, and these differences are partly responsible for the extensive number of medium formulations. Medium formulations are well reported in the art. In some embodiments, the cell culture medium comprises a basal medium.

In some embodiments, the porous membrane is inside a chamber comprising a compound characterized by an electrical modulation activity on blood brain barrier (BBB) of a brain region. In some embodiments, the compound comprises dextran, 2-[N-(7-nitrobenz-2-oxa-1,3-diazol-4-yl) amino]-2-deoxy-D-glucose (2-NBDG), sodium fluorescein, fluorescein isothiocyanate (FITC)-dextran, rhodamine, or any combination thereof.

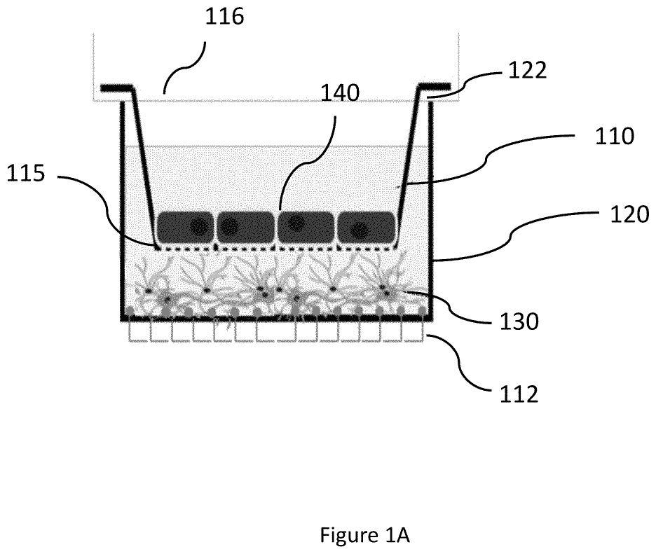

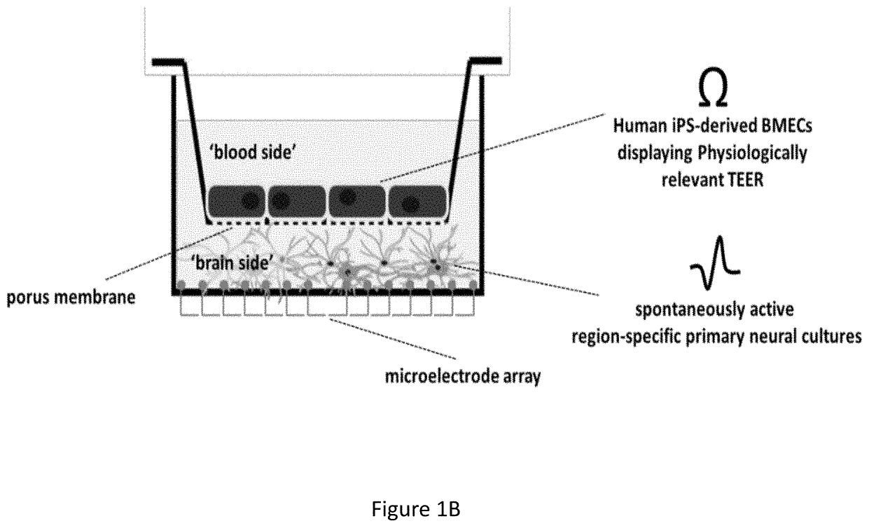

Reference is made to FIG. 1A showing a schematic illustration of the disclosed device in a non-limiting embodiment thereof.

The device may have a microelectrode 112. In some embodiments, the microelectrode comprises an array of microelectrodes. The array of microelectrodes 112 are arranged horizontally. Optionally, the array of microelectrodes 112 are parallel to each other. Optionally, the array of microelectrodes 112 are arranged along one direction. The microelectrode 112 may have cells 130 cultured on a surface of the microelectrode. In some embodiments, the cells comprise neural cells as described hereinabove.

The array of microelectrodes 112 and the porous membrane 155 are positioned in different planes parallel to each other. In some embodiments, the porous membrane 155 is positioned above the array of microelectrodes 112.

In some embodiments, the cells 130 are facing the lower surface of the porous membrane 115. Optionally, the lower surface of the porous membrane is devoid of cells.

In some embodiments, the porous membrane 115 and the microelectrode 112 may be inside a chamber. In some embodiments, the porous membrane 115 and the microelectrode 112 may be each inside a separate chamber (the porous membrane 115 inside chamber 110 and the microelectrode 112 inside chamber 120), and in fluid communication.

In some embodiments, the chamber 110 and the chamber 120 are configured to receive a fluid. The chamber 110 comprises an opening 116 configured to receive a fluid. The chamber 120 comprises an opening 122 configured to receive a fluid.

In some embodiments, the porous membrane 115 comprises an upper surface comprising a layer of cells 140. In some embodiments, the cells comprise endothelial cells. In some embodiments, the lower surface of the porous membrane 115, facing the cells 130, is devoid of endothelial cells.

In some embodiments, a device as described herein as provided in FIGS. 1A-B has an upper portion and a lower or bottom portion. In some embodiments, an upper portion (or upper chamber 110) comprises a membrane and cells. In some embodiments, an upper portion comprises a membrane and endothelial cells. In some embodiments, an upper portion comprises a membrane and cells and is devoid of a microelectrode and/or neural cells. In some embodiments, a lower or bottom portion (or lower chamber 120) comprises the microelectrode and the neural cells. In some embodiments, a lower or bottom portion comprises the microelectrode and cells and is devoid of a membrane and/or endothelial cells.

The term “chamber”, as used herein, means a natural or artificial at least partially enclosed space or cavity known to those of skill in the art. In some embodiments, the chamber comprises an opening.

In some embodiments, the upper chamber and the lower chamber have substantially identical dimensions and/or a substantially identical shape. Optionally, the chambers are configured to receive a fluid volume.

In some embodiments, the chamber has a cylindrical shape. In some embodiments, the chamber has a rectangular shape. In some embodiments, the chamber has a circular shape. In some embodiments, the chamber has a semicircular shape. In some embodiments, the chamber has an ellipsoid shape. In some embodiments, the chamber forms at least one “U” shape and/or at least one “T” shape. In some embodiments, the chamber is in a form of a channel.

In some embodiments, the chamber comprises a surface. In some embodiments, the surface is planar or non-planar. In some embodiments, the surface is facing the fluid volume. In some embodiments, the surface is configured to be in liquid communication with the fluid volume. In some embodiments, the surface is referred to an inner portion of at least one wall, wherein the inner portion is configured to face or to be in contact with the fluid volume. In some embodiments, the chamber comprises a plurality of surfaces.

In some embodiments, the microelectrode and the porous membrane are in contact. In some embodiments, the microelectrode and the porous membrane are not in contact or separate. In some embodiments, the cells cultured on the microelectrode are not in physical contact or communication with the cells cultured on the membrane. In some embodiments, the cells cultured on the microelectrode are in fluid communication with the cells cultured on the membrane. In some embodiments, the extracellular microenvironment of the cells cultured on the microelectrode is in fluid communication with the cells cultured on the membrane. In some embodiments, the extracellular microenvironment of the cells cultured on the membrane on the microelectrode is in fluid communication with the cells cultured on the microelectrode. In some embodiments, the extracellular microenvironment comprises a fluid as described hereinabove.

In some embodiments, the microelectrode comprises a tissue culture plate. In some embodiments, the tissue culture plate comprises a plurality of wells or 1 to 200 wells. In some embodiments, the tissue culture plate comprises one well. In some embodiments, the tissue culture plate comprises a plurality of wells. In some embodiments, each well or group of wells comprises a microelectrode, a porous membrane on top of the microelectrode and cells cultured on the microelectrode and the membrane.

In some embodiments, the microenvironment or an extracellular component of or secreted from a neural cell in contact or in fluid communication with the membrane. In some embodiments, a neural cell cultured on the microenvironment is in contact with the membrane but not with the cells cultured on the membrane. In some embodiments, a neural cell cultured on the microenvironment is in contact with a surface of the porous membrane devoid of the cells cultured on the membrane. In some embodiments, a membrane comprises at least two surfaces: a cell culturing surface and a surface facing a neural cell and/or in contact with a neural cell. In some embodiments, a membrane has two sides: a cell culturing side and a side facing a neural cell and/or in contact with a neural cell. In some embodiments, a membrane is in contact with a neuron.

In some embodiments, the microelectrode comprises a metal wire. In some embodiments, the microelectrode comprises a fiber. In some embodiments, the culture plate is attached to the top side of the electrode. In some embodiments, the microelectrode measures the membrane potential of the cells cultured on the microelectrode/the culture plate. In some embodiments, the microelectrode measures the action potential of neurons cultured on the microelectrode. In some embodiments, the microelectrode electrically stimulates the cells cultured on the microelectrode. In some embodiments, the microelectrode comprises a microelectrode array (MEA). In some embodiments, the microelectrode comprises a microelectrode array (MEA) plate.

In some embodiments, the membrane comprises a plurality of pores, wherein the shapes and sizes of the pores are highly controlled. In some embodiments, the diameter of each pore ranges from 0.1 micron (μm) to 10 microns (μm), 0.5 μm to 10 μm, 0.9 μm to 10 μm, 1 μm to 10 μm, 5 μm to 10 μm, 0.1 μm to 9 μm, 0.5 μm to 9 μm, 0.9 μm to 9 μm, 1 μm to 9 μm, 5 μm to 9 μm, 0.1 μm to 5 μm, 0.5 μm to 5 μm, 0.9 μm to 5 μm, or 1 μm to 5 μm, including any range therebetween. Each possibility represents a separate embodiment of the invention.

In some embodiments, the membrane is a semi-porous membrane. In some embodiments, the membrane is a porous membrane.

As used herein, the terms “pore”, “semi-porous” and “porous” refer to an opening or depression in the surface of the membrane. Porous membranes are well known and described in the art. Any suitable membrane can be used according to the present invention. Examples of suitable membranes include, but are not limited to, polyester or polycarbonate membranes. In some embodiments, the membrane is composed of polycarbonate. In some embodiments, the membrane is composed of polyester. In some embodiments, the membrane is coated with a substance prior to culturing the cells on the membrane. In some embodiments, the substance is a protein. In some embodiments, the substance is collagen. In some embodiments, the membrane is composed of collagen-coated polytetrafluoroethylene. In some embodiments, the electrical resistance of the membrane with the cells cultured on the membrane is at least 2000 Ω×cm². In some embodiments, the electrical resistance of the membrane with the cells cultured on the membrane is at least 4000 Ω×cm². In some embodiments, the electrical resistance of the membrane with the cells cultured on the membrane is at least 5000 Ω×cm². In some embodiments, the porous membrane is characterized by an electrical resistance of at least at least 2000 Ω×cm², at least 4000 Ω×cm², at least 5000 Ω×cm², at least 6000 Ω×cm², at least 7000 Ω×cm², at least 8000 Ω×cm², or at least 10000 Ω×cm², including any value therebetween. Each possibility represents a separate embodiment of the invention. In some embodiments, the electrical resistance is measured using a chop-stick electrode and a volt/ohm-meter.

In some embodiments, the cells cultured on the microelectrode comprise at least 90% neural cells. In some embodiments, the cells cultured on the microelectrode comprise at least 90% neural cells and devoid of endothelial cells. In some embodiments, the neural cells must comprise neurons. In some embodiments, the neurons express a neuronal marker. In some embodiments, the neurons express βIII-tubulin. In some embodiments, the neural cells comprise glial cells. In some embodiments, the glial cells express a glial cell marker. In some embodiments, the glial cells express glial fibrillary acidic protein. In some embodiments, the glial cells comprise astrocytes, oligodendrocytes, ependymal cells, microglia, or any combination thereof. In some embodiments, the neural cells comprise pericytes. In some embodiments, the neural cells comprise neurons, astrocytes, oligodendrocytes, ependymal cells, microglia, pericytes, or any combination thereof. In some embodiments, the neural cells are isolated from the brain. In some embodiments, the neural cells are isolated from a spinal cord. In some embodiments, the neural cells are isolated from brain cortex, striatum, hippocampus, cerebellum, spinal cord, or any combination thereof. In some embodiments, single brain region comprises one of brain cortex, striatum, hippocampus, cerebellum, spinal cord. In some embodiments, the neural cells comprise neurons. In some embodiments, the neural cells consist neurons. In some embodiments, the neural cells comprise neurons and neuroglia. In some embodiments, the neural cells are devoid of endothelial cells.

In some embodiments, the neural cells are derived from a primary cell culture. In some embodiments, the neural cells are derived from a single brain region. In some embodiments, the neural cells are isolated from a rat or a plurality of rats. In some embodiments, the neural cells are isolated from an embryo, or from a plurality of embryos. In some embodiments, the neural cells are isolated from an embryo at day 18 gestation. In some embodiments, the neural cells are isolated from a plurality of embryos at day 18 gestation. In some embodiments, the neural cells are isolated from a male mammal, a male rat, a male embryo, a male embryo at day 18 gestation, a female mammal, a female rat, a female embryo, a female embryo at day 18 gestation, or any combination thereof.

In some embodiments, the cells cultured on the membrane comprise at least 90% endothelial cells. In some embodiments, the cells cultured on the membrane comprise at least 90% endothelial cells and are devoid of neural cells. In some embodiments, the endothelial cells are of human origin. In some embodiments, the endothelial cells comprise brain microvascular endothelial cells (BMECs). In some embodiments, the endothelial cells form paracellular tight junctions. In some embodiments, the endothelial cells express an endothelial cell marker. In some embodiments, the endothelial cells express Pecam-1, Claudin-5, Occludin, Zona Occludens, Glut-1, Monocarboxylate transporter 8, or any combination thereof.

In some embodiments, the endothelial cells are derived from induced pluripotent stem cells (iPSCs). In some embodiments, the iPSCs are derived from fibroblasts. In some embodiments, the endothelial cells are induced pluripotent stem cell-derived brain microvascular endothelial cells (iBMECs). In some embodiments, the iBMECs are derived by culturing iPSCs in medium with growth factors in the absence of serum.

### The Method

In some embodiments, provided herein is a method for mimicking or modeling the BBB. In some embodiments, provided herein is a method for mimicking or modeling the BBB in-vitro. In some embodiments, provided herein is a method for in-vitro mimicking or modeling the BBB of a specific brain region.

As used herein, the term “in-vitro” refers to an artificial environment and to processes or reactions that occur within an artificial environment. In vitro environments can consist of, but are not limited to, test tubes and cell culture. The term “in-vivo” refers to the natural environment (e.g., an animal or a cell) and to processes or reaction that occur within a natural environment.

In some embodiments, provided herein is a method for assessing the action-potential modulation activity of a compound comprising contacting the compound with neural cells and measuring the membrane's potential compared to a steady state membrane potential. In some embodiments, the compound is a test compound.

As used herein, the term “test compound” refers to any chemical entity, pharmaceutical, drug, and the like that is a candidate for use to treat or prevent a disease, illness, sickness, or disorder of bodily function. Test compounds comprise both known and potential therapeutic compounds. A test compound can be determined to be therapeutic by screening using the screening methods of the present invention. In some embodiments of the present invention, test compounds are agents (e.g. chemical or biological agents) that function in the CNS or agents that are useful in investigating the regulation or regulating the integrity and/or function of the blood brain barrier. In some embodiments, test compounds are “drugs with unknown blood brain barrier passive capability.” As used herein, the term “drugs with unknown blood brain barrier passive capability” refers to drugs whose ability to cross the blood brain barrier is unknown. In some embodiments, “drugs with unknown blood brain barrier passive capability” are known to have therapeutic activity, while in other embodiments, the therapeutic activity of the drug is unknown.

In some embodiments, the devices and methods disclosed herein are for use in research applications, such as drug screening. In some embodiments, the devices and methods disclosed herein are for use in screening of drugs that function in the brain and thus optimally cross the blood brain barrier (e.g., drugs that act on the central nervous system). In some embodiments, brain endothelial cells as described herein are placed in a device of embodiments of the present invention. In some embodiments, endothelial cells are contacted with test compounds. Test compounds are assayed for their ability to cross the endothelial cell monolayer. Test compounds (e.g., drugs) can be assayed for their ability to cross the endothelial cell monolayer using a variety of methods. In some embodiments, membrane properties and integrity is measured by TEER values. In some embodiments, test compounds are assayed for their ability to cross the cell monolayer. In some embodiments, test compounds may be further screened for their ability to exert a biological effect on the cells contained in the device.

In some embodiments, provided herein is a method for assessing the ability of a compound to modulate the permeability of the endothelial layer, comprising applying the compound on the endothelial layer and: (a) measuring the membrane potential; and/or (b) measuring the amount of compound within the neural cells compartment (the bottom compartment of the device.

In some embodiments, provided herein is a method for measuring resistance of a blood-brain-barrier (BBB), comprising measuring resistance of the membrane within the device, thereby measuring the resistance of the BBB. In some embodiments, provided herein is a method for identifying a compound having an electrical modulation activity on BBB of a brain region, comprising measuring resistance of the membrane within the device in a steady state (control, without the compound), applying the compound onto the cells cultured on the membrane and/or on the neural cells on the microelectrode, and assessing whether the compound increased or decreased the resistance or the potential.

In some embodiments, provided herein is a method for measuring resistance of a blood-brain-barrier (BBB) in a single brain region, comprising measuring resistance of the membrane within the device comprising or consisting neural cells of a single brain region, thereby measuring the resistance of the BBB in a single brain region. In some embodiments, provided herein is a method for measuring a change in resistance of a blood-brain-barrier (BBB) in a single brain region, comprising first measuring resistance of the membrane within the device comprising or consisting neural cells of a single brain region, applying a compound or a physical stimulus (such as temperature, irradiation, etc.), second measuring resistance of the membrane within the device comprising or consisting neural cells of a single brain region, and computing the difference in potential/resistance between first measuring and second measuring, thereby identifying a change in resistance of a blood-brain-barrier (BBB) in a single brain region.

In some embodiments, a change in resistance of a blood-brain-barrier is modulating resistance of a blood-brain-barrier. In some embodiments, resistance is interchangeable with potential or electric potential. In some embodiments, resistance, potential or electric potential is measured on the membrane. In some embodiments, resistance, potential or electric potential of the membrane is modulated by the neural cells, the endothelial cells or both.

In some embodiments, provided herein is a method for determining an ability of a compound to affect permeability of a BBB, comprising administering the compound in an upper portion of the device, and then measuring resistance of the membrane within the device, thereby determining the ability of a compound to affect the permeability of the BBB.

In some embodiments, provided herein is a method for determining an ability of a compound to affect permeability of a BBB, comprising administering the compound in an upper portion of the device, and then measuring resistance of the membrane within the device, thereby determining the ability of a compound to affect the permeability of the BBB. In some embodiments, a compound penetrating into the lower portion of the device and affecting the action potential of neurons is identified as a consequence of a measurable difference in resistance between steady state and post treatment with the compound. In some embodiments, a composition enabling the penetration of a compound into the lower portion of the device and affecting the action potential of neurons is identified as a consequence of a measurable difference in resistance between steady state and post treatment with the composition comprising the compound. In some embodiments, provided herein is a method for determining an ability of a compound to affect permeability of a BBB is in a single brain region.

In some embodiments, provided herein is a method for determining an ability of a compound to traverse a BBB, comprising administering the compound in or on an upper portion of the device, and then measuring amount of the compound in the lower portion of the device.

In some embodiments, a compound as described herein is labeled. In some embodiments, a compound as described herein comprises an organic moiety. In some embodiments, a compound as described herein is a candidate neurostimulator. In some embodiments, a compound as described herein is a candidate BBB permeability modulator.

In some embodiments, provided herein is a method for determining an ability of a compound to modulate neuronal activity across a BBB, comprising administering the compound in or on an upper portion of the device, and then measuring an action potential of the neural cells (the membrane's resistance), thereby determining the ability of a compound to modulate neuronal activity across the BBB. In some embodiments, neuronal activity is neuronal electrical activity or action potential. In some embodiments, a compound triggers neuronal activity. In some embodiments, a compound enhances neuronal activity. In some embodiments, a compound blocks or inhibits neuronal activity.

In some embodiments, administering the compound or the composition in or on an upper portion of the device comprises administering the compound or the composition onto the endothelial cells as described herein.

In some embodiments, provided herein is a method for determining a neurotoxic potential of a compound across a BBB, comprising administering the compound in an upper portion of the device, and then assessing cell death of the neural cells or a decrease in action potential of the neural cells, thereby determining the neurotoxic potential of a compound across the BBB.

In some embodiments, action potential of the neural cells is measured by measuring the membrane's resistance. In some embodiments, neurons of the neural cells are in contact with the membrane. In some embodiments, modulation of action potential of the neural cells correlates with modulation of the membrane's resistance.

In some embodiments, endothelial cells comprise iBMECs. In some embodiments, endothelial cells comprise modified iBMECs. In some embodiments, iBMECs comprise modified iBMECs. In some embodiments, modified iBMECs are obtained by culturing iBMECs for at least 2, 5, 8, 10, 15, or 24 hours in the presence of a composition comprising an extra-cellular environment of neural cells or cultured within an upper portion of the device as described herein. Each possibility represents a separate embodiment of the present invention. In some embodiments, modified iBMECs are iBMECs matured or cultured in the presence of factors secreted from neural cells (neural cells extracellular environment).

In some embodiments, provided herein is a method for differentiating induced pluripotent stem cells (iPSCs) to brain microvascular endothelial cells (BMECs), wherein the iPSCs are cultured in serum free medium, thereby differentiating the iPSCs to BMECs. In some embodiments, the BMECs are cultured within an upper portion of the device, and wherein the electrical resistance of the membrane is at least 1000, 2000, 3000, 4000, or 5000 Ω×cm².

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

There is currently no in vitro platform that is able to model human Central nervous system-penetrability in a region-specific manner. The inventors developed a novel approach that will allow the generation of neurovascular unit (NVU) platforms that are specific for the cortex, striatum, hippocampus, cerebellum and spinal cord.

In order to generate brain-region specific NVU platforms, the inventors propose to create a dual compartment system in which iBMECs will be cultured on a transwell insert as previously described. Brain-region-specific BBB properties will be achieved by co-culturing induced pluripotent stem cell-derived brain microvascular endothelial cells (iBMECs) on top of primary rat neural cultures, containing region-specific astrocytes and neurons (FIGS. 1A-B). By co-culturing these cell types the inventors aim to recreate the intercellular interactions that cause these differences in vivo. The primary neural cells will be grown on microelectrode array (MEA) plates to allow real-time monitoring of neuronal activity. Combined, these platforms will be able to simultaneously monitor both, molecule blood brain barrier (BBB) penetrability and the effect of molecule on neuronal activity.

### Methods

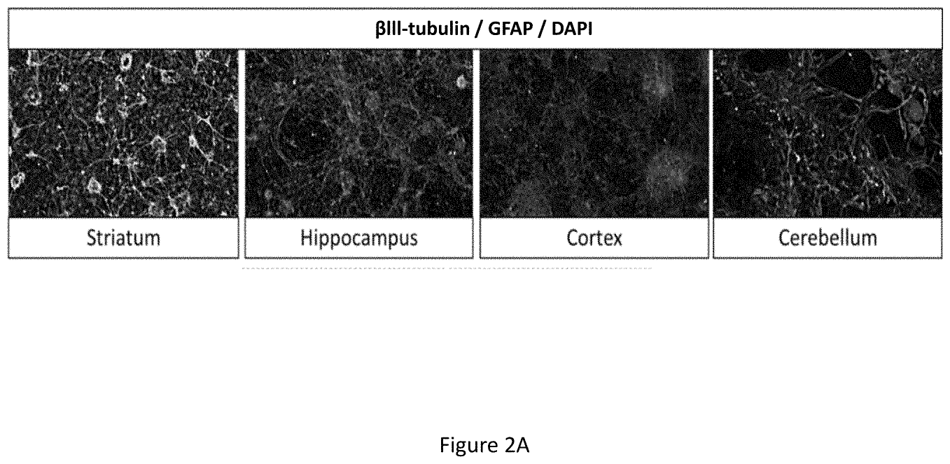

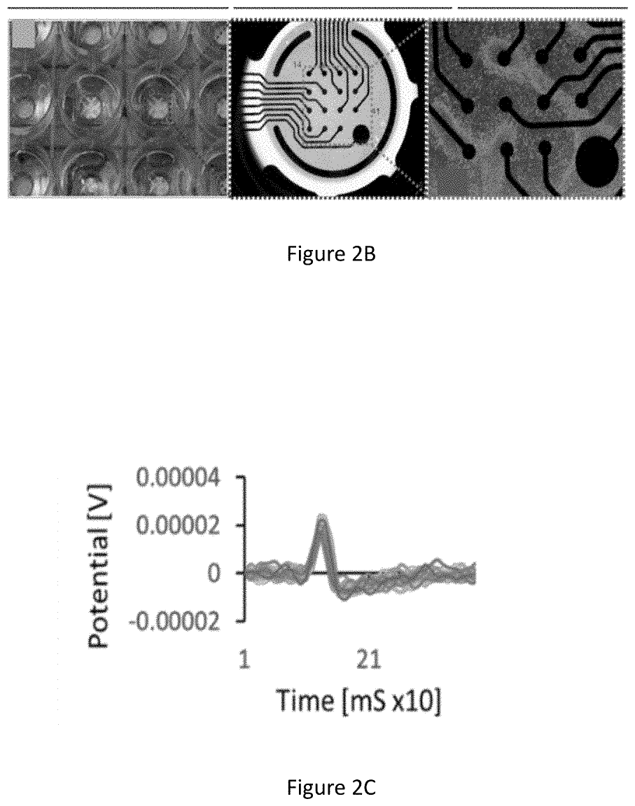

In order to obtain region-specific primary neural cultures, E18 rat embryos were extracted from a pregnant female. Embryos were dissected and several regions of the CNS were separated including cortex, striatum, hippocampus, cerebellum and spinal cord. The regions were enzymatically dissociated into single cells before seeded pre-coated MEA plates and petri dishes, in which they were cultured in Neurobasal medium (Sigma) for two weeks. In order to validate the various primary cell cultures, the cells were stained for the neuronal marker βIII-tubulin and the glial marker GFAP (FIGS. 2A-C). Fibroblasts were reprogrammed into induced pluripotent stem cells (iPSCs) using non-integrating episomal plasmids. Three clones from each iPSC line were characterized by immunocytochemistry for the pluripotency markers NANOG/TRA160, OCT4/SSEA4 and TRA181/SOX2, as well as by Alkaline phosphatase (AP) activity and karyotype analysis. iPSCs were then differentiated into iBMECs. On day 8 of differentiation, cells were enzymatically dissociated into single cell suspensions and seeded on pre-coated transwell inserts. 24 hours post seeding, iBMEC inserts were placed on top of primary neural cultures derived from the different CNS regions where they were cultured for three additional days. iBMECs expressed markers for paracellular tight junctions, and following an optimized differentiation protocol, they physiologically relevant demonstrated transendothelial electrical resistance (TEER) levels.

### Results

iBMECs that were co-cultured on top of the various region-specific primary neural cultures, were assessed by transendothelial electrical resistance (TEER), an electrophysiological hallmark of barrier properties. These results indicate that iBMECs that are co-cultured on top of neural cells display increased barrier properties, which were made possible recreating the inter-cellular cross talk between the neural cultures and iBMEC monolayers. The differential TEER measurements demonstrate the expected variations between the BBB across different neural cultures. These results demonstrate the feasibility of the inventors developed approach.

### Example 2

### Generation of Brain Region-Specific Neurovascular Units for Prediction of Drug Penetrability Across the Human BBB

### Approach

In order to generate brain-region specific NVU platforms, the inventors propose to create a dual compartment system in which iBMECs will be cultured on a transwell insert as previously described. Brain-region-specific BBB properties will be achieved by co-culturing iBMECs on top of primary rat neural cultures, containing region-specific astrocytes and neurons (FIGS. 1A-B). By co-culturing these cell types the inventors aim to recreate the intercellular interactions that cause these differences in vivo. The primary neural cells will be grown on microelectrode array (MEA) plates to allow real-time monitoring of neuronal activity. Combined, these platforms will be able to simultaneously monitor both, molecule BBB penetrability and the effect of molecule on neuronal activity.

### Methods

In order to obtain region-specific primary neural cultures, E18 rat embryos were extracted from a pregnant female. Embryos were dissected and several regions of the CNS were separated including cortex, striatum, hippocampus, cerebellum and spinal cord. The regions were enzymatically dissociated into single cells before seeded pre-coated MEA plates and petri dishes, in which they were cultured in Neurobasal medium (Sigma) for two weeks. In order to validate the various primary cell cultures, the cells were stained for the neuronal marker βIII-tubulin and the glial marker GFAP (FIG. 2).

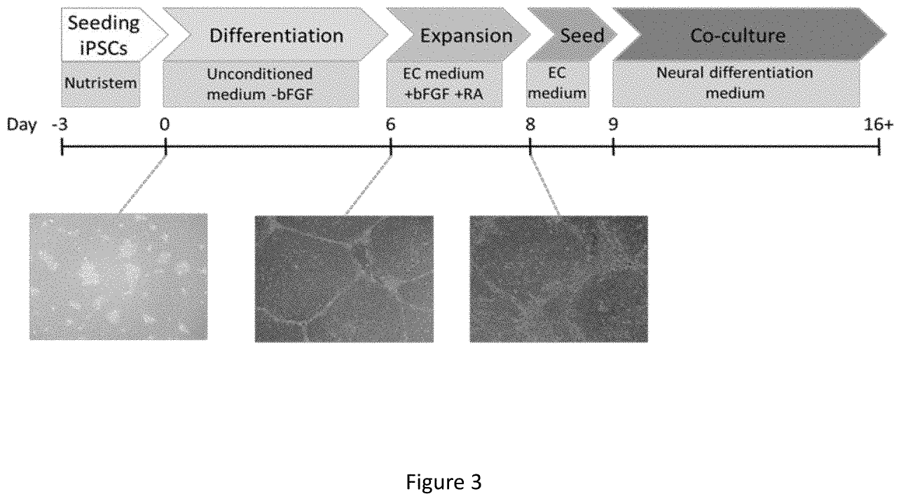

iPSCs were differentiated into iBMECs as previously described. On Day 8 of differentiation, cells were enzymatically dissociated into single cell suspensions and seeded on precoated transwell inserts. 24 hours post seeding, iBMEC inserts were placed on top of primary neural cultures derived from the different CNS regions (FIG. 3) where they were cultured for three additional days.

### Results

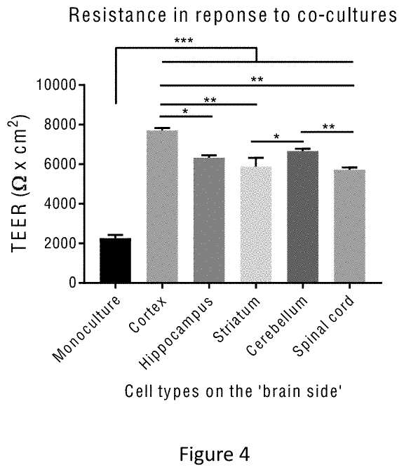

iBMECs that were co-cultured on top of the various region-specific primary neural cultures, were assessed by transendothelial electrical resistance (TEER), an electrophysiological hallmark of barrier properties (FIG. 4). These results indicate that iBMECs that are co-cultured on top of neural cells display increased barrier properties, which were made possible recreating the inter-cellular cross talk between the neural cultures and iBMEC monolayers. The differential TEER measurements demonstrate the expected variations between the BBB across different neural cultures. These results demonstrate the feasibility of the inventors developed approach.

Functional assessment of five molecules across the iBMEC monolayer. Permeability should be differential across different brain regions according to the known in vivo differences.

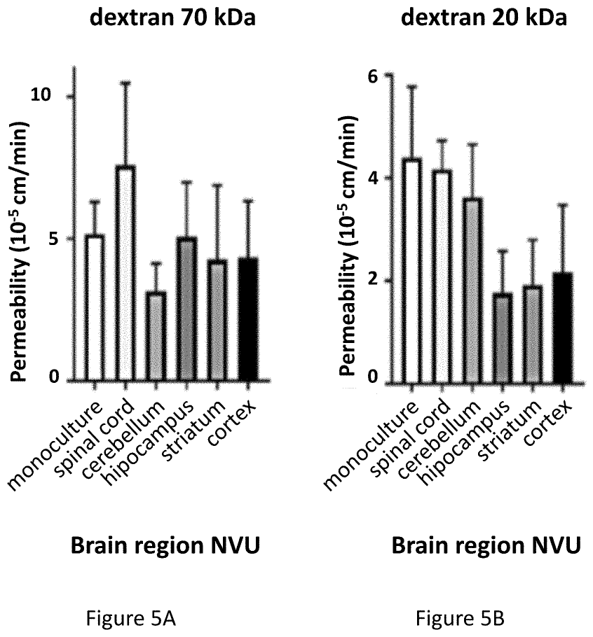

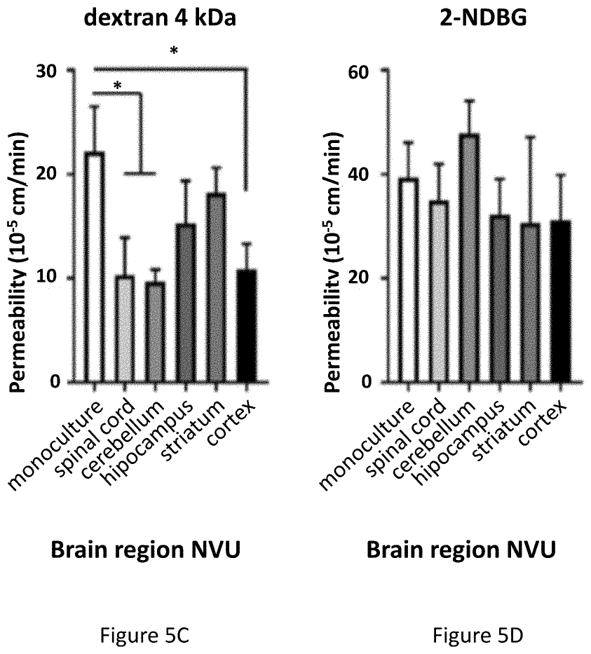

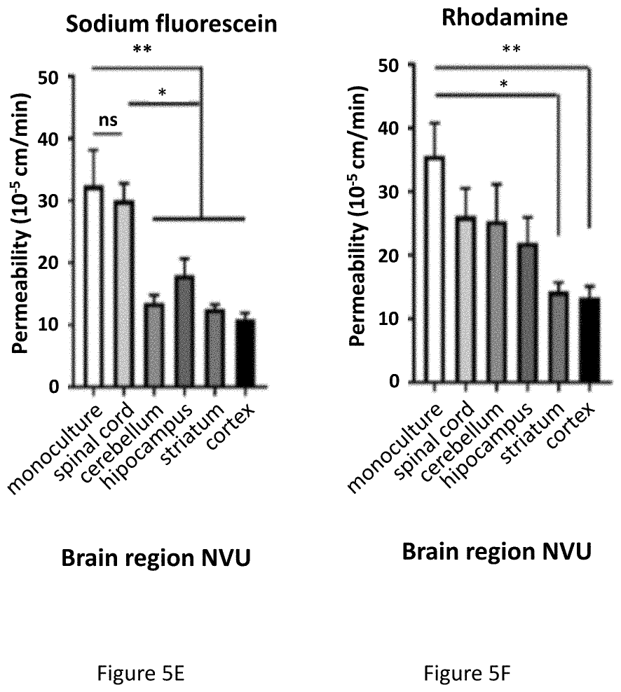

In order to functionally test the performance of the different NVUs, the inventors have tested the blood-to-brain permeability of six molecules including fluorescent tracers of different molecular weight (dextran-TRITC of 70 kDa, dextran-FITC of 20 kDa, dextran-FITC 4 kDa, sodium fluorescein (332 Da), the efflux pump substrate Rhodamine 1,2,3 (480 Da) and the glucose analogue fluorescent deoxyglucose derivative (2-NBDG). Experiments were performed as previously described by adding each molecule to the top chamber of the transwells, and samples were collected from the bottom chamber after 15, 30, 45 and 60 min. Fluorescence was measured using a plate reader and calculated into concentrations using a calibration curve with known concentrations. Next, the inventors plotted the chart and extracted the slope for each transwell. Slopes from empty transwells were subtracted from the obtained value and the results were used to calculate the permeability, expressed as 10-5 cm/min. The inventor's results show that the large molecular tracers (dextran of 70 kDa and 20 kDa) poorly crossed all iBMECs with no significant differences between the various NVUs (FIGS. 5A-B). These tracers are equivalent in size to large molecules, thus it is not surprising that they were unable to efficiently cross all iBMECs, including the monocultures. The smaller tracer, dextran 4 kDa showed the expected higher permeability, with significant differences between the monoculture and NVUs of the spinal cord, cerebellum and the cortex. The permeability of 2-NDBG, an analogue of glucose, was not different between the NVUs. This is in line with the expression of the glucose transporter 1 (GLUT1) throughout the BBB and iBMECs, where it is responsible for glucose transport into the CNS. The smaller tracer, sodium fluorescein was significantly more permeable in the monoculture compared to the NVUs of the cortex, cerebellum, striatum and hippocampus. Importantly, significance differences in permeability were also observed between spinal cord and other NVUs, confirming the expected in vivo differences. These results were similar to the permeability of the small tracer Rhodamine. Overall, the permeability results showed a brain region effect only with the smaller tracers, which is expected due to the high TEER that was achieved in all iBMECs including the monocultures.

### Discussion

The BBB is formed as a multicellular NVU, which tightly restricts the passage of molecules from the blood circulation into the CNS. As such, the BBB presents a major obstacle in the delivery of neurological drugs into the CNS as it restricts the passage of most FDA-approved molecules. In vivo models represent the physiological properties of the BBB and can be used to model the heterogeneity of the BBB across different regions, however, animal models are poor predictors of human BBB penetrability. Consequently, human based models have been widely used to study BBB properties. In the last decade, iBMECs were introduced as a scalable and robust endothelial cell source, which demonstrated physiologically relevant BBB properties including the expression of TJ proteins, endothelial markers and functional transport systems. Importantly, iBMECs display barrier properties that reach levels similar to the in vivo BBB. Despite these crucial characteristic, today's in vitro systems of the BBB fail to represent the heterogeneity across different regions of the CNS.

Here, the inventors have developed an approach in which iBMECs are co-cultured with primary neural cells derived from various regions of the CNS including cortex, striatum, hippocampus, cerebellum and spinal cord. The inventors results demonstrate that the recreated NVU platforms display expected variations permeability. These results provide the basis for this patent application.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device comprising:
i. a microelectrode comprising cells cultured on a surface of said microelectrode; and
ii. a porous membrane comprising an upper surface comprising cultured cells and a lower surface; wherein:
said porous membrane is positioned above said microelectrode and said cells cultured on said microelectrode are facing said lower surface of said porous membrane.

2. The device of claim 1, wherein said lower surface is devoid of said cultured cells of said upper surface, optionally wherein said cells cultured on said microelectrode comprise at least 90% neural cells, optionally wherein said cells cultured on said microelectrode comprise at least 90% neural cells from a single brain region, optionally wherein said neural cells are selected from the group consisting of: cortex neural cells, striatum neural cells, hippocampus neural cells, cerebellum neural cells, and spinal cord neural.

3.-5. (canceled)

6. The device of claim 1, wherein said cultured cells on said porous membrane comprise at least 90% endothelial cells.

7. The device of claim 1, wherein said cultured cells on said porous membrane comprise at least 90% brain microvascular endothelial cells.

8. The device of claim 1, wherein said cultured cells on said porous membrane comprise at least 90% induced pluripotent stem cell-derived brain microvascular endothelial cells (iBMECs).

9. The device of claim 1, wherein said cells cultured on said microelectrode comprise glial cells, and optionally wherein said microelectrode comprises a microelectrode array (MEA) plate.

10. (canceled)

11. The device of claim 1, wherein said porous membrane is characterized by an electrical resistance of at least 5000 Ω×cm², optionally wherein said porous membrane and said microelectrode are inside a chamber comprising a fluid, optionally wherein said porous membrane and said microelectrode are each inside a separate chamber and in fluid communication, and optionally wherein said fluid comprises a cell culture medium.

12.-14. (canceled)

15. A method for measuring resistance of a blood-brain-barrier (BBB), comprising measuring resistance of said porous membrane within the device of claim 1, thereby measuring the resistance of said BBB.

16. A method for measuring resistance of a blood-brain-barrier (BBB) in a single brain region, comprising measuring resistance of said membrane within the device of claim 2, thereby measuring the resistance of said BBB in a single brain region.

17. A method for determining an ability of a compound to affect permeability of a BBB, comprising administering said compound in an upper portion of said device of claim 1, and then measuring resistance of said porous membrane within the device of, thereby determining the ability of a compound to affect the permeability of said BBB.

18. A method for determining an ability of a compound to affect permeability of a BBB in a single brain region, comprising administering said compound in an upper portion of said device of claim 2, and then measuring resistance of said membrane within the device of claim 2, thereby determining the ability of a compound to affect the permeability of said BBB in a single brain region.

19. A method for determining an ability of a compound to traverse a BBB, comprising administering said compound in an upper portion of said device of claim 1, and then measuring amount of said compound in the lower portion of said device of, thereby determining the ability of a compound to traverse said BBB.

20. A method for determining an ability of a compound to traverse a BBB in a single brain region, comprising administering said compound in an upper portion of said device of claim 2, and then measuring amount of said compound in the lower portion of said device of, thereby determining the ability of a compound to traverse said BBB in a single brain region.

21. A method for determining an ability of a compound to modulate neuronal activity across a BBB, comprising administering said compound in an upper portion of said device of claim 1, and then measuring an action potential of said neural cells of, thereby determining the ability of a compound to modulate neuronal activity across said BBB.

22. A method for determining an ability of a compound to modulate neuronal activity across a BBB in a single brain region, comprising administering said compound in an upper portion of said device of claim 2, and then measuring an action potential of said neural cells of, thereby determining the ability of a compound to modulate neuronal activity across said BBB in a single brain region.

23. A method for determining a neurotoxic potential of a compound across a BBB, comprising administering said compound in an upper portion of said device of claim 2, and then assessing cell death of said neural cells of, thereby determining the neurotoxic potential of a compound across said BBB.

24. A method for determining a neurotoxic potential of a compound across a BBB in a single brain region, comprising administering said compound in an upper portion of said device of claim 2, and then assessing cell death of said neural cells of, thereby determining the neurotoxic potential of a compound across said BBB in a single brain region.

25. A method for generating modified iBMECs, comprising culturing iBMECs for at least 10 hours in the presence of a composition comprising an extra-cellular environment of neural cells or cultured within an upper portion of said device of claim 1.

26. A method for differentiating induced pluripotent stem cells (iPSCs) to brain microvascular endothelial cells (BMECs), wherein said iPSCs are cultured in serum free medium, thereby differentiating said iPSCs to BMECs.

27. The method of claim 26, wherein said BMECs or iBMECs are cultured within an upper portion of a device comprising:
i. a microelectrode comprising cells cultured on a surface of said microelectrode; and
ii. a porous membrane comprising an upper surface comprising cultured cells and a lower surface; wherein:
said porous membrane is positioned above said microelectrode and said cells cultured on said microelectrode are facing said lower surface of said porous membrane, and wherein the electrical resistance of said membrane is at least 5000 Ω×cm².
